(19) United States
(12) Patent Application Publication
(10) Pub. No.: US 2013/0303921 A1
(43) Pub. Date: Nov. 14, 2013

(54) System and Method for Measurement of Physiological Data with Light Modulation

(71) Applicants: Lut Hey CHU, Hong Kong SAR (CN); Ka Cheung KWOK, Hong Kong SAR (CN); Chun ZHANG, Hong Kong SAR (CN)

(72) Inventors: Lut Hey CHU, Hong Kong SAR (CN); Ka Cheung KWOK, Hong Kong SAR (CN); Chun ZHANG, Hong Kong SAR (CN)

(73) Assignee: Hong Kong Applied Science and Technology Research Institute Company Limited, Hong Kong SAR (CN)

(21) Appl. No.: 13/469,094

(22) Filed: May 11, 2012

(51) Int. Cl. A61B 6/00

(52) U.S. Cl. 600/473; 600/476

(57) ABSTRACT

The present invention discloses a device for measuring physiological data of a subject. It comprises a light modulation unit, an optical detection unit and a signal processing unit. The present invention can operate in an active mode or a passive mode to measure a subject's heart rate, respiratory information, haemoglobin level, cardiac output or oxygen saturation of the blood, etc. Fourier Transform based lock-in technique is used to detect the physiological signals reliably even when the signal is weak. In addition, ambient light can be used as the light source to complete the measurement.

### FIELD OF INVENTION

This invention relates to a measurement device, and in particular a non-contact type device for measuring the physiological data of a subject.

### BACKGROUND OF INVENTION

Medical measuring devices are widely used to monitor vital parameters of our body. A photoplethysmograph (PPG) is an optical signal obtained by a device that transmits optical rays to a subject's tissue and analyzes the light absorption profile in order to non-invasively determine the physiological data of the subject.

A pulse oximeter is an example of such measuring devices. It is used to indirectly monitor the oxygen saturation of a person's blood, using the ratio of red to infrared light absorption from the pulsating components verse the non-pulsating components as the blood flows through the artery vein. By using different light sources with different wavelengths, other PPG devices can be designed to measure different physiological signals.

However, most of the PPG devices are contact-type devices that make direct contact with the subject's skin. In order to secure a good and firm contact, the device may apply pressure to clamp or stick (adhere to) on a certain part of the subject's body. This may make the subject uncomfortable, and in cases like monitoring the physiological data of a baby, it may even agitate the baby, rendering the measurement inaccurate. If the clamping pressure is not sufficient, then there may be slippage between the device and skin when in use. This will cause deviation to the measurement. A non-contacting PPG device, however, can take the measurement without having to touch the subject. And in some situations, the subject may even be unaware that a measurement has taken place. The subject can be in a calm or peaceful state of mind, or carry on his or her normal activity. The resultant measurements can then reflect the actual physiological condition of the subject in a normal state. On the other hand, the absorption signal(s) received at the detector is much weaker for non-contact type operation, as the signal strength decreases dramatically with the increase of the optical distance traversed to the detector.

### SUMMARY OF INVENTION

In the light of the foregoing background, it is an object of the present invention to provide an alternate device and method to measure the physiological data with improved sensitivity and design.

Accordingly, the present invention, in one aspect, is a device for measuring physiological data of a subject. The device comprises: (a) a light modulation unit; (b) an optical detection unit; and (c) a signal processing unit. The light modulation unit generates at least one modulated light signal by modulating at least one light source at one or more predefined frequency. Each of the modulated light signals has a different wavelength and irradiates onto the subject. It is then regulated by a physiological signal of the subject to generate a composite spectrophotometric light signal. The optical detection unit receives the composite spectrophotometric light signal and converts it into an electrical signal. The signal processing unit is adapted for converting the electrical signal into a digital signal and obtaining a frequency spectrum of the digital signal. It further tracks at least one dominant spectral peak of the frequency spectrum, and recognizes at least one minor spectral peak at the frequency spectrum from the vicinity of the respective dominant spectral peak. Lastly, it determines the physiological data based on the measurements of the one or more dominant spectral peak(s) and the corresponding minor spectral peak(s).

In an exemplary embodiment of the present invention, the light modulation unit comprises an electronic circuit that turns the light source on and off at the predefined frequency. In another embodiment, the light modulation unit comprises a wheel. The wheel is opaque to light and comprises at least one hollow area so that when it rotates at a predetermined frequency, light rays emitted from the one or more light sources are intermittently transmitted and blocked. In this way, the light rays are modulated. The modulation frequency depends on the predetermined frequency of the rotating wheel as well as the number of hollow areas in the rotating wheel for that particular light source.

In another exemplary embodiment, the device emits a red light that is modulated at a predefined frequency and an infra-red light that is modulated at another predefined frequency.

In a further embodiment, the light source is ambient light and the hollow area of the wheel is coupled to a color filter that only allows light with certain wavelength to pass through. In one embodiment, red or infra-red filters are used to generate the desirable modulated light signals.

In another implementation, the light modulation unit comprises a static disc with at least one color filter thereon and a wheel with at least one hollow area. The static disc is coupled to the wheel. The wheel rotates at a predetermined frequency and the static disc is stationary in which the respective color filter and the corresponding hollow area align along an axis intermittently when the wheel rotates so that light emitted from the light source is intermittently transmitted and blocked and thus is modulated at the predefined frequency.

In another embodiment, the signal processing unit further comprises an analog-to-digital convertor, a central processing unit and memory. The processing unit executes an embedded program stored in the memory for determining the physiological data. The physiological data includes heart rate, heart rate variability, respiratory information, haemoglobin level, arterial stiffness index, cardiac output, oxygen saturation of the blood, or any combination thereof.

In yet another embodiment, the device operates in a non-contact type of operation. It further comprises an optical collection unit for directing the composite spectrophotometric light signal to the optical detection unit. The optical collection unit may comprise (a) one or more lens, (b) one or more mirrors, (c) waveguides, (d) optical fibers or (e) any combination of the above. The optical detection unit comprises a device selected from a photodiode, a photomulitplier tube, a CMOS array and a CCD array.

According to another aspect of the present invention, it is an apparatus for measuring physiological data of a subject that comprises the same light modulation unit, the optical detection unit, and the signal processing unit as said above. The apparatus may further comprise the optical collection unit mentioned before. The main difference is that ambient light first impinges onto the subject and then is regulated by the physiological signal of the subject. The regulated light signal is reflected to the light modulation unit. The latter further modulates the regulated light signal using the apparatus and techniques mentioned before. The resultant composite spectrophotometric light signal is sent to the optical detection unit and it is processed in the same way by the signal processing unit as mentioned before.

In another aspect, the present invention is a method for determining physiological data of a subject. The method comprises the steps of: (a) generating at least one modulated light signal by modulating at least one light source at at least one predefined frequency, each at least one modulated light signal having a different wavelength; (b) detecting a composite spectrophotometric light signal and converting the composite spectrophotometric light signal into an electrical signal, whereby the composite spectrophotometric light signal is generated when a physiological signal of the subject is regulated by one or more modulated light signal; (c) converting the electrical signal into a digital signal; (d) obtaining a frequency spectrum of the digital signal; (e) tracking at least one dominant spectral peak at the frequency spectrum, each dominant spectral peak corresponding to each at least one predefined frequency; (f) recognizing one or more minor spectral peak(s) at the frequency spectrum from the vicinity of the respective dominant spectral peak; and (g) determining the physiological data from the respective dominant spectral peak and the corresponding minor spectral peak(s).

In yet another embodiment, the determining step (g) of the method further comprises the step of computing the average of a minor spectral peak at the upper side band and another minor spectral peak at the lower side band of the respective dominant spectral peak.

There are many advantages to the present invention. Firstly, the present invention uses Fourier Transform based lock-in technique to calculate the results. The modulation frequency and phase are self-tracked. There is no need to obtain reference signal for demodulation and the corresponding filtering circuits can then be omitted. Thus the size of the device can be reduced and so the device is more portable. Moreover, the sensitivity of the present invention is much higher and hence it can be used in a non-contact manner.

Another advantage of the present invention is that the device can function well even when ambient light is used as the light source. The present invention is non-disturbing to the users in this way, especially for measuring physiological data of a baby.

### BRIEF DESCRIPTION OF FIGURES

FIG. 1a is a block diagram illustrating the basic idea of the overall system operating in active mode.

FIG. 1b is a block diagram illustrating the basic idea of the overall system operating in passive mode.

FIG. 2 shows the waveforms of the carrier signal, message signal and the modulated signal respectively according to one embodiment of the present invention.

FIG. 3 shows various components of the light absorption profile when light penetrates to the blood artery.

FIG. 4a shows an actual waveform of the detected signal in time domain according to one embodiment of the present invention.

FIG. 4b shows the spectral magnitude of the detected signal after Fourier Transform according to one embodiment of the present invention.

FIG. 5 is a first implementation of the present invention according to an embodiment.

FIG. 6 is a second implementation of the present invention according to an embodiment.

FIG. 7 is a third implementation of the present invention according to an embodiment.

FIG. 8 shows various wheels with different configurations according to an embodiment of the present invention.

FIG. 9 is a fourth implementation of the present invention according to an embodiment.

FIG. 10 shows another configuration of the wheel modulation according to one embodiment of the present invention.

FIG. 11 shows a simple circuit that implements the optical detection unit and the signal processing unit of the present invention in one embodiment.

FIG. 12 shows a flow chart of the Fourier Transform based lock-in detection algorithm of the present invention.

FIG. 13 illustrates several embodiments that the present invention can be configured to operate at different environments.

### DETAILED DESCRIPTION OF THE PREFERRED EMBODIMENTS

As used herein and in the claims, “comprising” means including the following elements but not excluding others. “Couple” or “connect” refers to electrical or mechanical coupling or connection either directly or indirectly via one or more electrical or mechanical means unless otherwise stated. Furthermore, “lock-in” refers to a technique of modulating a signal at a relatively high frequency and then capturing it in a narrowband close to the modulation frequency with locked phase so as to reduce wideband noise. In one embodiment, it further means for detecting a small light signal against a bright background.

The embodiments described herein disclose inventive ideas of capturing and analyzing physiological data of a subject and can be implemented in a number of ways. In particular, an active mode configuration and a passive mode configuration are disclosed below. Based on the teaching of this disclosure, other configurations or variations can also be realized by those skilled in the art but they would still fall in the scope of the present invention.

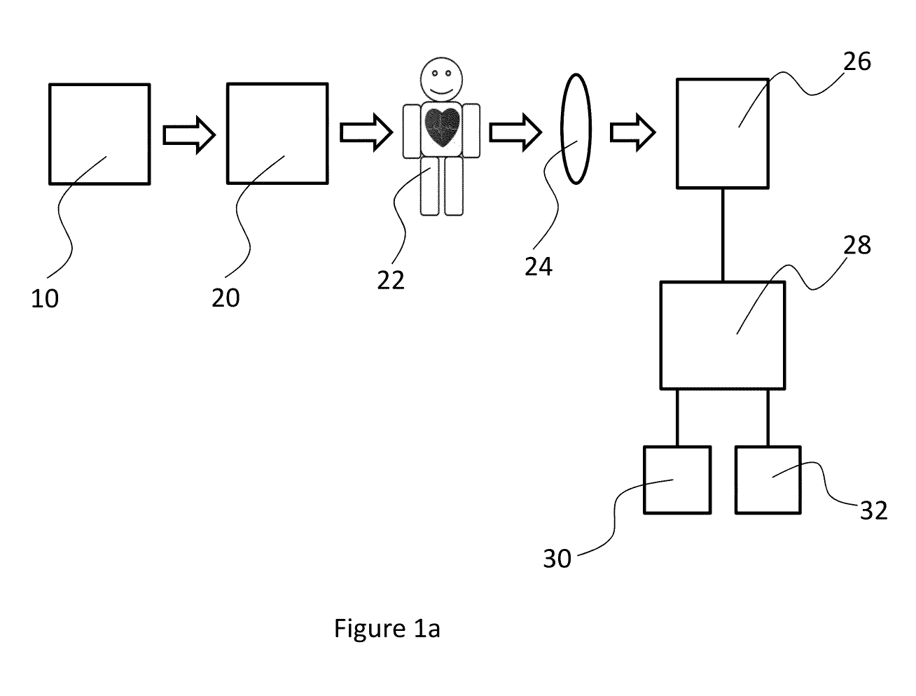

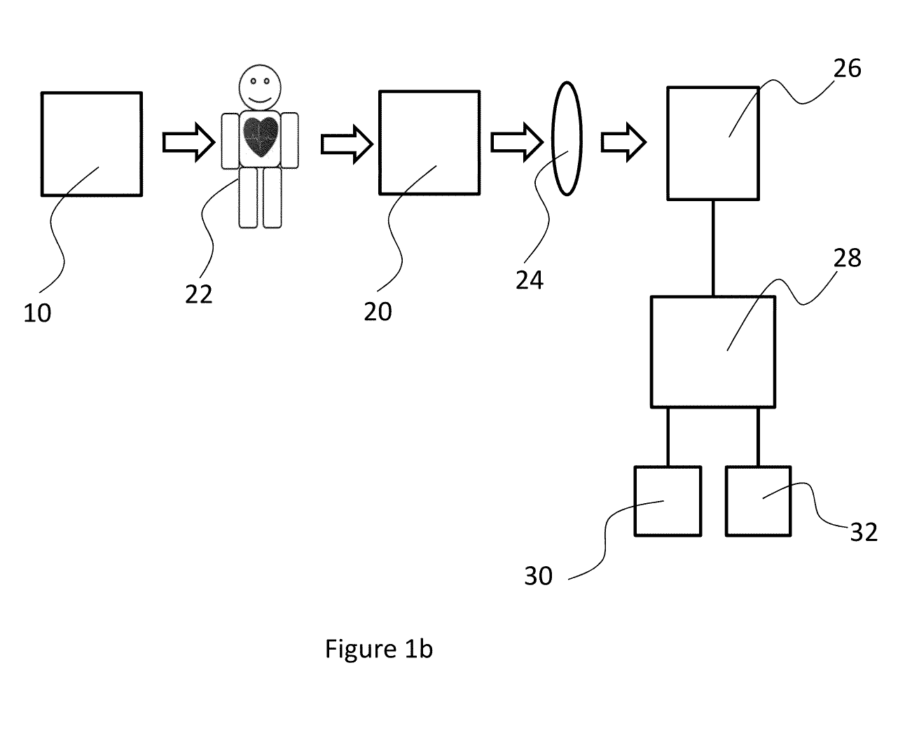

Referring now to FIG. 1a, a block diagram illustrating the basic idea of the overall system operating in active mode is shown. It comprises at least one light source 10 that emits at least one light beam to a light modulation unit 20. The latter modulates one or more light beams to generate at least one modulated light signal. The one or more modulated light signals then irradiates onto the subject 22, which can be a human or animal. This modulated light signal(s) is/are further regulated by a physiological signal that is intended to be measured, resulting in a composite spectrophotometric light signal. The latter can then be concentrated by an optical collection unit 24 before it enters an optical detection unit 26. The optical detection unit 26 converts the light signal into electrical signal which is then processed by a signal processing unit 28. After calculating the physiological data, the results can be displayed by a display unit 30 and/or stored in an external device 32. Depending on what physiological data to be measured, the number of modulated light signals required is different. It should be noted that the display unit 30, the external device 32 and the optical collection unit 24 are optional components that may or may not be required to implement the present invention, depending on the application environment and user requirements.

FIG. 1b shows another embodiment of the present invention that operates in a passive mode. The difference between FIG. 1a and FIG. 1b is that the light modulation unit 20 is positioned behind the subject 22 in FIG. 1b whereas it is in front of the subject 22 in FIG. 1a. In other words, light beam(s) is/are first modulated by the light modulation unit 20 before regulated by the physiological signal of the subject in active mode (FIG. 1a) whereas light is first regulated by the physiological signal and then modulated in the passive mode (FIG. 1b). Other than that, the optical flow and the principle of operations are the same. It should be noted that in FIG. 1b, the light source 10 may be ambient light. Thus, an apparatus that implements a passive mode configuration of the present invention may not need to include a component that generates light beam(s) for it to operate.

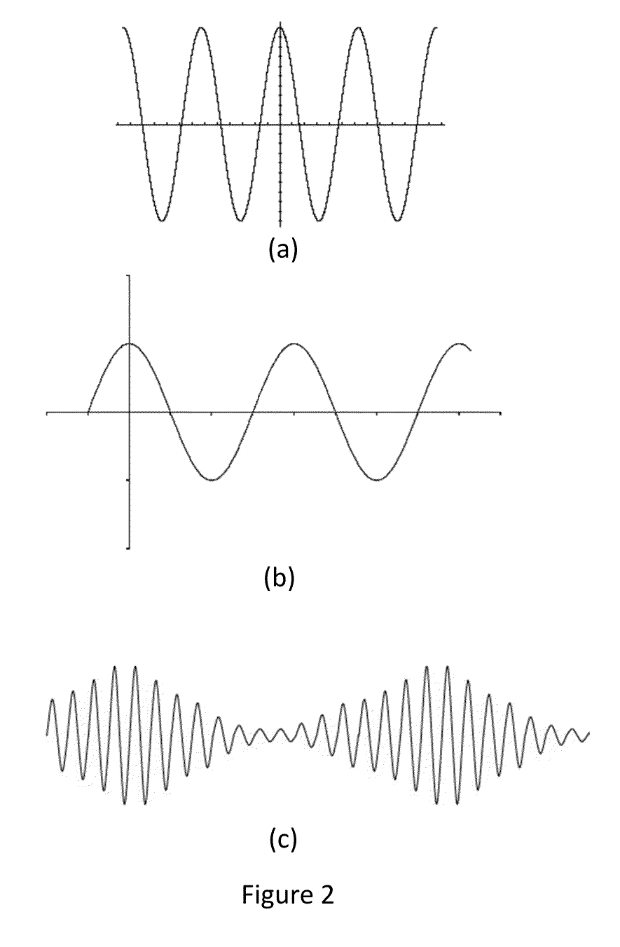

In the following paragraphs, an oximeter operating in the active mode configuration is used as an exemplary embodiment to illustrate this invention. This specific application makes use of the fact that the red light and infra-red light have different absorption rates when they pass through or are reflected from the body of the subject 22. Hence, a red light and an infra-red light are used as the light inputs in this case. In one embodiment, the wavelength of the red light is 660 nm while that of the infra-red light is 940 nm. Using the ratio of red to infra-red light absorption from the pulsating components of the blood volume changes, the oxygen saturation can be obtained. In a further embodiment, the light modulation unit 20 modulates the red light and infra-red light at different predefined frequencies, e.g. 80 Hz and 300 Hz respectively, to obtain two modulated light signals. In other embodiments, the predefined frequencies can be of any value but they should not be a multiple of one another. The two modulated light signals may be of different waveforms (e.g. square wave, sinusoidal wave), depending on the preference and the requirements. The modulated red light and the modulated infra-red light can be regarded as carrier signals with frequencies 80 Hz and 300 Hz respectively. For illustrating purpose, the modulated red light and the modulated infra-red light are taken as sinusoidal wave as shown in FIG. 2a. The equations of the modulated red light and the modulated infra-red light are:

R=A₁ cos 2πf₁t; and

IR=A₂ cos 2πf₂t;

where R and IR are the modulated red light and the modulated infra-red light respectively;

A₁ and A₂ are the amplitudes of red light and infra-red light respectively; and

f₁ and f₂ are the modulation frequencies of the red light and the infra-red light respectively.

The two carrier signals are then emitted onto the subject 22. In a transmission pulse oximeter (TPO), the two carrier signals are typically emitted to the subject's appendage (e.g., finger, ear lobe or nasal septum) on one side and the optical detection unit 26 receives attenuated carrier signals on the other side. For a reflectance pulse oximeter (RPO), the optical detector unit 26 detects the reflected light of the tissue and hence is placed at the same side as the light modulation unit 20. Moreover, the present invention can operate in a contact mode or a non-contact mode. In the contact mode, the light modulation unit 20 and/or the optical detector unit 26 may touch the skin of the subject while in the non-contact mode, these two units do not make contact to the subject's skin.

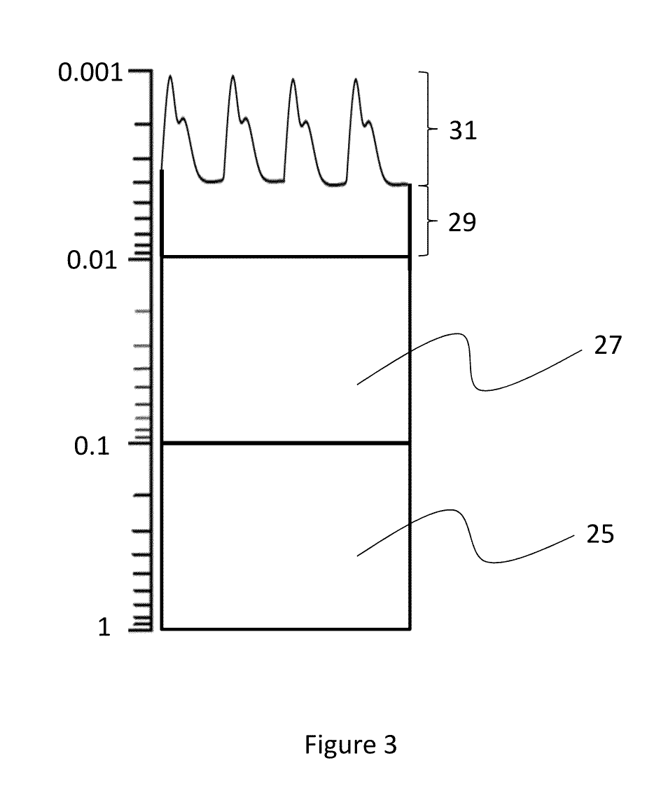

When the modulated light signals irradiate onto the artery tissue of the subject 22, the intensities of the modulated light signals are attenuated by the tissue, the bone structure, and also the pulsatile arterial blood. The amount of attenuation depends on the wavelength of the incident light, and is different for red light or infra-red light as well as the oxygen saturation level of the blood. FIG. 3 shows, in one embodiment, various components of the signal detected by the optical detection unit 26. As shown in FIG. 3, a large part of the light signal (absorption 25) is absorbed by the skin, bone and tissue of the subject 22. The absorption 27 in the middle of the figure is due to the venous blood. The upper part of figure shows the absorption due to the arterial blood. This absorption can be divided into two components: the absorption due to non-pulsatile arterial blood (which is referred as the DC part 29) and the absorption due to pulsatile arterial blood (which is referred as the AC part 31). The waveform of the AC part 31 has a dominant sinusoidal component with frequency around 0.5-4 Hz. This is the pulsation frequency of the subject 22. For simplicity and easy demonstration, the AC part 31 (pulsation wave) can be approximated by a sinusoidal waveform as shown in FIG. 2b. As a result, the equations of the pulsation waves of red light and infra-red light can be written as:

RP=δ₁ cos 2πf₀t; and

IRP=δ₂ cos 2πf₀t; eq. (1)

where RP and IRP are the pulsation waves of red light and the pulsation wave of infra-red light respectively;
δ₁ and δ₂ are the amplitudes of the pulsation wave of red light and the pulsation wave of infra-red light respectively; and
f₀ is the pulsation frequency of the tissue bed.

In the non-contact mode of operation, the composite spectrophotometric light signal received by the optical detector unit 26 is substantially weaker. This can be observed in the following table which compares the signal strengths between contact-type and non-contact type operation in one experimental results:

TABLE 1

| | Transmission-mode | Reflectance-mode |
|---|---|---|
| Contact-type | AC/DC = ~0.1 | AC/DC = ~0.01 |
| Non-contact-type | AC/DC = ~0.01 | AC/DC = ~0.001 or less |

As shown in table 1, the pulsation signal magnitude in non-contact type operation is 10 times lower than that of contact type operation in this experiment. Hence, it calls for a novel approach to recover the pulsation signal in a reliable and accurate manner

To boost the signal strength, an optical collection unit 24 is usually used to collect the composite spectrophotometric light signals and guide the same to the optical detection unit 26, as illustrated in FIGS. 1a and 1b. The optical collection unit 24 may comprises (a) one or more lens, (b) one or more mirrors, (c) waveguides, (d) optical fibers or (e) any combination of the above thereof.

When a modulated light signal irradiates onto the subject 22, it is attenuated or regulated by the pulsation waves. The net result is an amplitude-modulated signal whereby the modulated light signals are the carriers while the pulsation waves are the messages which are of much lower frequencies than that of the carrier waves. After amplitude modulation, a waveform as shown in FIG. 2c will be obtained. The light signal transmitted through or reflected from the subject 22 is referred as a composite spectrophotometric light signal (i.e. amplitude modulated signal) which is generated when a physiological signal (i.e. the pulsatile arterial blood) of the subject 22 is regulated by the modulated light signal. As for the oximeter application, red light and infra-red light are used. Thus the composite spectrophotometric light signal has the following equation:

$$\begin{matrix}S = ( 1 + RP ) R + ( 1 + IRP ) IR \\ = ( 1 + {δ}_{1} cos 2 π {f}_{0} t ) {A}_{1} cos ( 2 π {f}_{1} t + {ϕ}_{1} ) + \\ ( 1 + {δ}_{2} cos 2 π {f}_{0} t ) {A}_{2} cos ( 2 π {f}_{2} t + {ϕ}_{2} )\end{matrix} \qquad eq . ( 2 )$$

where S is the composite spectrophotometric light signal;
A₁ and A₂ are the amplitudes of red light and infrared light respectively;
f₁ and f₂ are the modulation frequencies of the red light and infrared light respectively; and
φ₁ and φ₂ are the phase difference between the pulsation wave and the modulation wave of red light and infra-red light respectively.

The composite spectrophotometric light signal is received by the optical detection unit 26 directly or through the optical collection unit 24. It is then converted into an electrical signal by the optical detection unit 26. Conventional measuring devices may demodulate the composite signal and then calculate the physiological data in time domain. Such process requires synchronization between the composite signal and the demodulated signal and thus a reference source is needed. The present invention use a Fourier Transform (FT) based lock-in technique to track the carrier signals and also to determine the physiological data in frequency domain. No reference source is needed. The tracking and analyzing algorithm is performed within the signal processing unit 28. In one embodiment, the signal processing unit 28 is an integrated circuit comprising an analog to digital convertor which converts the electrical signal from the optical detection unit 26 into a digital signal, a central processing unit (CPU), and memory that stores an embedded program. In a further embodiment, the signal processing unit 28 computes the Fourier Transform on the digital signal to obtain its frequency spectrum. In the embodiment of an oximeter application, the detected signal may be approximated by the equation below with the assumption that the modulated waves and the pulsation waves are pure sinusoidal.

S=(1+δ₁ cos 2πf₀t)A₁ cos(2πf₁t+φ₁)+(1+δ₂ cos 2πf₀t)A₂ cos(2πf₂t+φ₂) eq. (3)

This equation can be re-written to show the spectral components,

S=δ₁A₁ cos(2π(f₁−f₀)t+φ₁)/2+A₁ cos(2πf₁t+φ₁)+δ₁A₁ cos(2π(f₁+f₀)t+φ₁)/2+δ₂A₂ cos(2π(f₂−f₀)t+φ₂)/2+A₂ cos(2πf₂t+φ₂)+δ₂A₂ cos(2π(f₂+f₀)t+φ₂)/2 eq. (4)

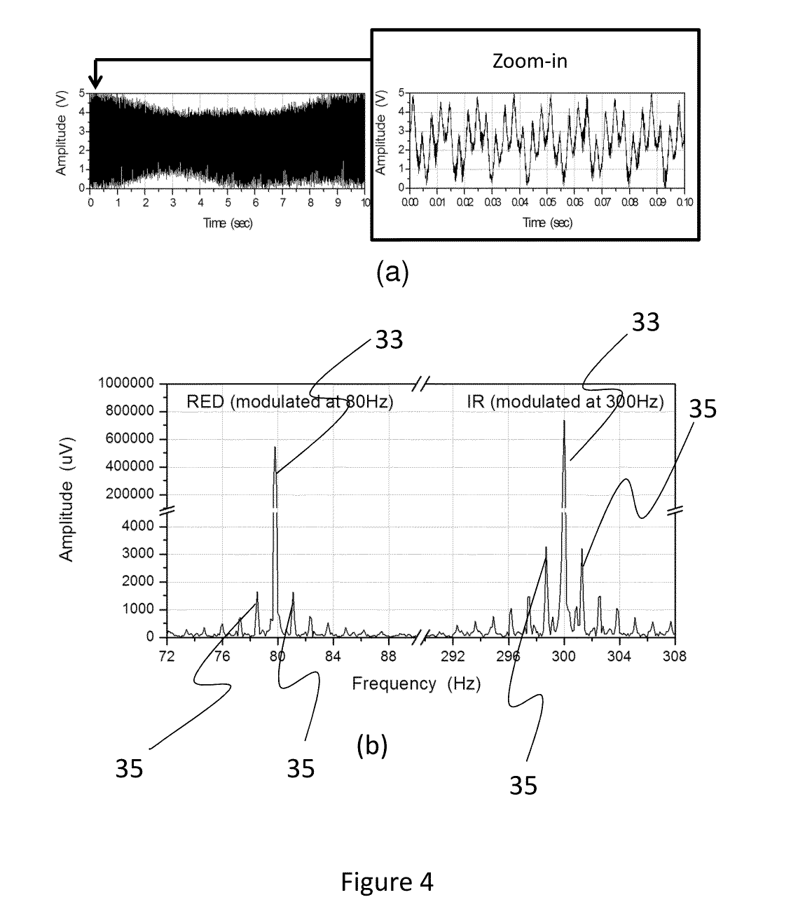

As shown in eq. (4), there are six components in the frequency spectrum of S as there are six different frequencies in the equation, corresponding to frequencies (f₁−f₀), f₁, (f₁+f₀), (f₂−f₀), f₂ and (f₂+f₀) respectively. As δ₁ and δ₂ are substantially less than unity, the value S is dominated by the second term and the fifth term of eq. (4). These two terms correspond to frequencies f₁ and f₂. i.e. the modulation frequencies of the red light and the infra-red light respectively. As a result, the frequency spectrum has two dominant spectral peaks with magnitude A₁ and A₂ respectively. They can easily be searched and located by the signal processing unit 28. The signal processing unit 28 further tracks the magnitudes and phases of these two modulation frequencies from one time frame to another. This is referred as Fourier Transform (FT) based lock-in and thus a reference signal is not needed for demodulation. After tracking the dominant spectral peaks, the signal processing unit 28 recognizes the minor spectral peaks from the vicinity of the dominant spectral peaks. The minor spectral peaks correspond to the frequencies (f₁−₀), (f₁+f₀), (f₂−f₀) and (f₂+f₀), and their magnitudes are much smaller than that of the dominant spectral peaks (please refer to FIG. 4 and its description below). The signal processing unit 28 then determines the physiological data from the dominant spectral peaks and the minor spectral peaks. In one embodiment, the ratio of ratio (RoR) is calculated by the following equation:

RoR=[A_{(f1±f0)}/A_{f1}]/[A_{(f2±f0)}/A_{f2}] eq. (5)

where A_{(f1±f0)} and A_{(f2±f0)} are the amplitudes of the minor spectral peaks near the dominant spectral peaks of the red light and the infra-red light respectively; and

A_{f1} and A_{f2} are the amplitudes of the dominant spectral peaks of the red light and the infra-red light respectively.

The blood oxygen saturation can then be calculated from RoR with pre-determined calibration.

In general, using the spectral magnitude value of a single minor spectral peak near the corresponding dominant spectral peak is sufficient to obtain an accurate oxygen saturation rate. However, if a higher accuracy is needed, the averaging of the two minor spectral peaks near the corresponding dominant spectral peak can be calculated.

In reality, the pulsation waves are not pure sinusoidal. Therefore, the frequency spectrum may not contain only six peaks but other higher order harmonics as well. Referring now to FIG. 4a, an actual waveform of the detected signal in time domain is shown according to one embodiment of the present invention. The right side of the figure is a zoom-in view of a fraction of the detected signal on the left. FIG. 4b shows the spectral magnitude of the detected signal after Fourier Transform. As seen, there are two dominant spectral peaks 33 and many minor spectral peaks around both the upper side band and the lower side band of each of the dominant spectral peaks. In an embodiment, the first minor spectral peak 35, either from the upper side band or from the lower side band is used for calculation in eq. (5). In another embodiment, the average of the two first minor spectral peaks 35 is used. In a further embodiment, spectral peaks from higher order harmonics are used for determining the oxygen saturation level of the blood. Unlike conventional measuring devices which use time division multiplex (TDM), the present invention adopts frequency division multiplex (FDM). In TDM, the receiver is susceptible to a wideband noise. However, the present invention adopts the FDM approach and uses a digital lock-in technique on the Fourier spectrum so that the modulation frequency and the phase are self-tracked. As such, the signal can be captured in a narrowband close to the modulation frequency (the dominant spectral peak 33) and thus less noise enters the system. Therefore, the reference signal and complex filtering circuits can be omitted, making it possible to be implemented in a simple and compact module.

When the oxygen saturation is calculated, the signal processing unit 28 may send the result to the display unit 30 for outputting the result. The signal processing unit 28 may be also connected to an external device such as a memory storage unit, a smart phone, a printer, a computer, etc. for further processing or analysis.

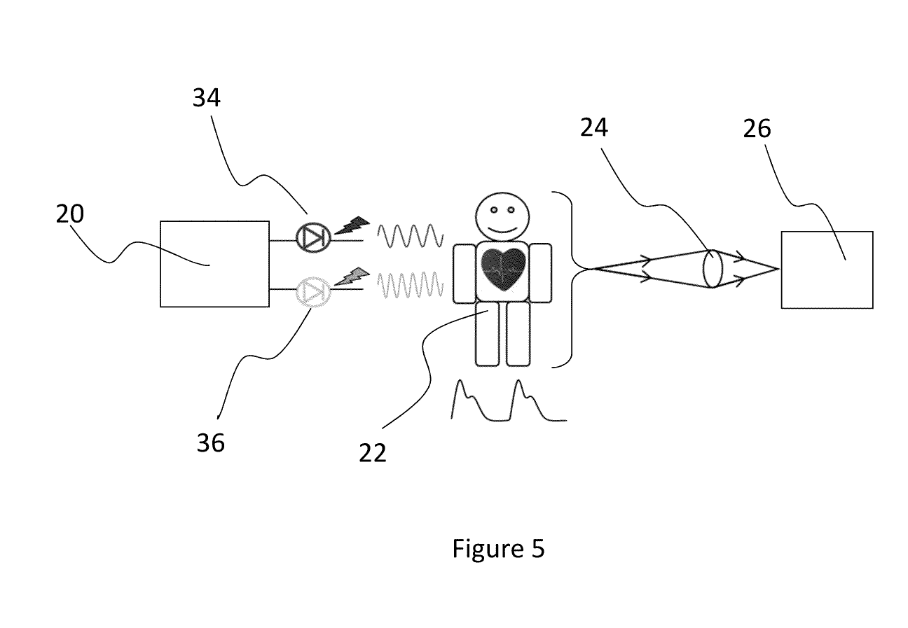

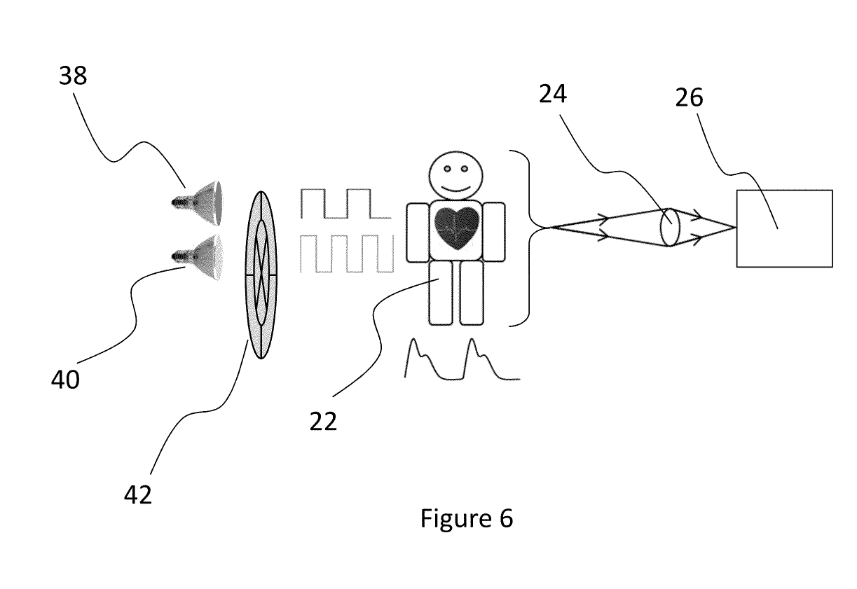

Now turning to FIG. 5, a first implementation of the present invention is shown according to an embodiment. In this implementation, a red LED and an infra-red LED are used as the light sources. The light modulation unit 20 comprises an electronic circuit that turns the red LED and the infra-red LED on and off at the predefined frequencies. In one embodiment, they are 200 Hz and 300 Hz respectively. The modulated light signals emitted by the red LED and the infra-red LED can be square waves, sinusoidal waves, etc., depending on the requirements and the modulation method used by the light modulation unit 20. In an embodiment, Transistor-Transistor-Logic (TTL) is used to generate square waves for modulating the light sources. In other embodiments, other methods are used to generate sinusoidal, triangular or other wave shapes. As previously described, the modulated signals then irradiate onto the subject 22 and transmit through the subject 22 or reflected from the subject 22. The composite spectrophotometric light signals are then collected and detected for calculation. The basic concept is same as mentioned before.

FIG. 6 shows the second implementation of the present invention according to an embodiment. A high power red lamp 38 and a high power infra-red lamp 40 are used as the light sources for the situation that a high intensity is needed. In one embodiment, the halogen lamps covered with red or infrared filters are used. However, the light modulation unit 20 does not modulate the light signals electrically through the modulation of power current supplying to the light emitters as many of the high power lamps cannot switch on or off fast enough at frequency higher than 20 Hz. Rather, the modulation unit 20 uses a wheel 42 to modulate the light signals mechanically. The wheel 42 is opaque to light and thus blocks the red light and the infra-red light from passing through. Furthermore, the wheel 42 comprises at least one hollow area for allowing light to pass through. The wheel 42 is rotated at a predetermined frequency, e.g. 100 Hz, 150 Hz, etc. By having different number of the hollow areas and different arrangement of shapes and positions of the hollow areas, the red light and the infra-red light can be modulated at different predefined frequencies. When the wheel 42 is rotating, the light rays emitted by the light sources are intermittently transmitted and blocked so as to perform modulation mechanically. The predefined frequencies are integer multiples of the predetermined frequency. In one embodiment, there are two hollow areas in the outer circumference equally spaced around the wheel 42 for light from the red lamp 38 to pass through, and another three hollow areas equally spaced in a circumference just below the two hollow areas in the outer circumference for light from the infra-red lamp 40 to pass through. When the wheel rotates at 100 Hz, a modulated red light signal of 200 Hz and a modulated infra-red light signal of 300 Hz are generated. In addition, by have different shapes of the hollow areas, the light signals passing through can have different waveforms, e.g. square, triangle and sinusoidal, etc. After modulation, the light goes through the same processing steps as described above. The calculation step is performed in in the signal processing unit 28.

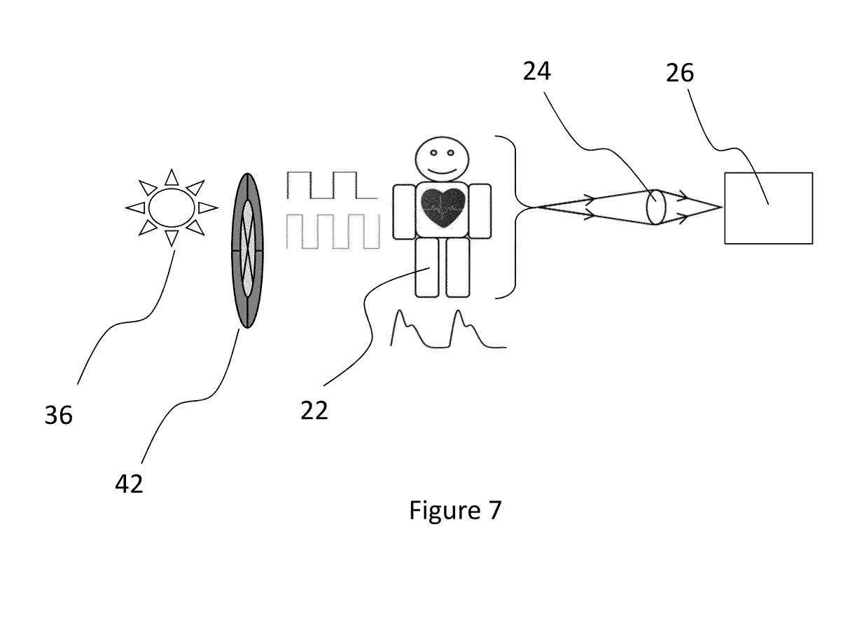

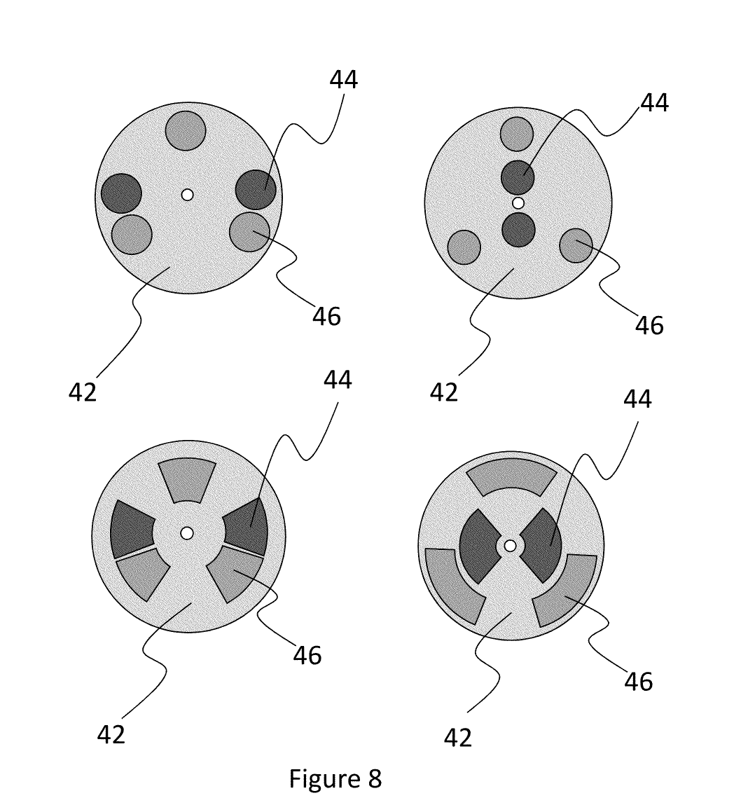

Referring to FIG. 7, a third implementation of the present invention is shown. A single light source 36 is used. This light source contains both the red light and infra-red light in its spectrum. In one embodiment, it can be the ambient light. In a further embodiment, it can be sun light. In yet another embodiment, it can be the indoor ambient light, which uses florescent lamp, light bulb, etc. for illumination. Since the ambient light may contain additional color components other than the red light and infra-red light. The wheel 42 is modified not only to generate modulated light signals but also to filter out unwanted color components. In one embodiment, the wheel 42 is opaque to light and comprises at least one hollow area. The hollow area is coupled to a color filter for allowing light with certain wavelength to pass through. The wheel 42 rotates at a predefined frequency and thus modulates the wanted light signals at a predetermined frequency. The wheel 42 can have different number of the hollow areas and different arrangement of shapes and positions of the hollow areas. FIG. 8 shows various wheels 42 with different configurations. For all these four configurations, there are two hollow areas coupled to red light filters 44. They are symmetrically positioned relative to the center of the wheel. There are also three hollow areas coupled to infra-red light filters 46. They are equally distributed angularly with constant radial displacement from the wheel center; and they do not overlap with the red light filters 44. When the wheel rotates at 100 Hz, it generates a 200 Hz modulated red light signal and a 300 Hz modulated infra-red light signal. FIG. 8 demonstrates the principle of generating one or more modulated color light signals at different predefined frequencies. There can be many variations on the design of this wheel. For example, the wheels 42 can have different numbers and shapes of red light filters 44 and infra-red light filters 46, and the positions thereof can be arranged differently. The type, shape, number and position of the filters are highly flexible, depending on the modulation frequencies required, shapes of the waveform and the light rays of certain colors to be permitted to transmit through. Going back to FIG. 7, it can be seen that the ambient light is filtered and modulated before it irradiates onto the subject 22. After filtering and modulation, the subsequent steps are the same as described above, i.e. collecting the composite spectrophotometric light signal, converting the same into an electrical signal, etc.

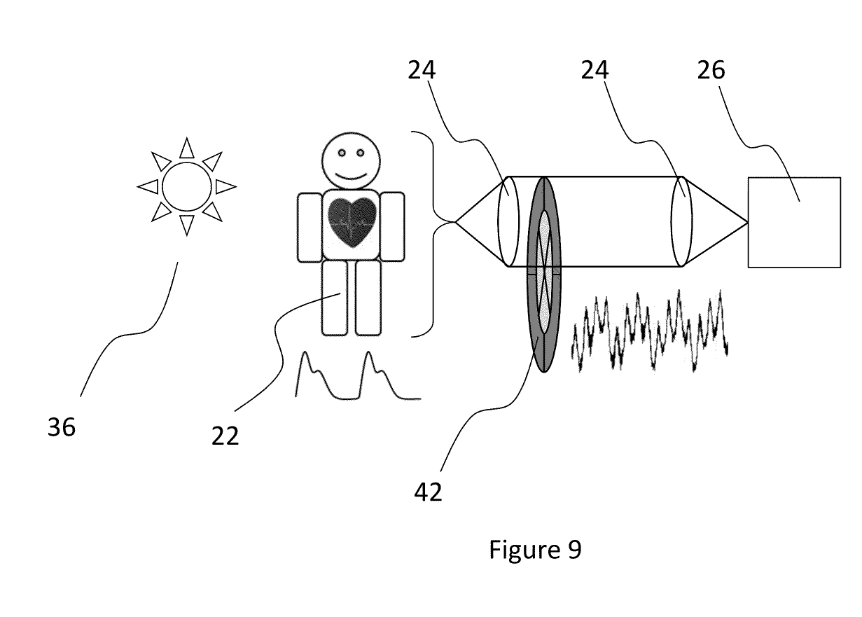

Both FIGS. 5, 6 and 7 discuss the active mode configurations of the present invention. An embodiment of the passive mode operation is shown in FIG. 9, which is also referred as the fourth implementation of the present invention. This implementation is similar to the third implementation as described above. The light source 36 and the configuration of the wheel 42 are substantially the same as the third implementation. The main difference is that light rays first irradiate on the subject and is regulated by the physiological signal of the subject. As a result, a spectrophotometric light signal is generated. This spectrophotometric light signal further passes through the wheel 42, which filters out unwanted color light and also modulates the desired color light in a predefined frequency. The wheel 42 then turns the spectrophotometric light signal into a composite spectrophotometric light signal as defined in the previous paragraphs. Afterwards, the subsequent processing steps are the same as in the active mode operation and are not repeated here. In one embodiment, one or more optical collection units 24 are installed in front/back of the wheel 42 for collecting and guiding the light passing through.

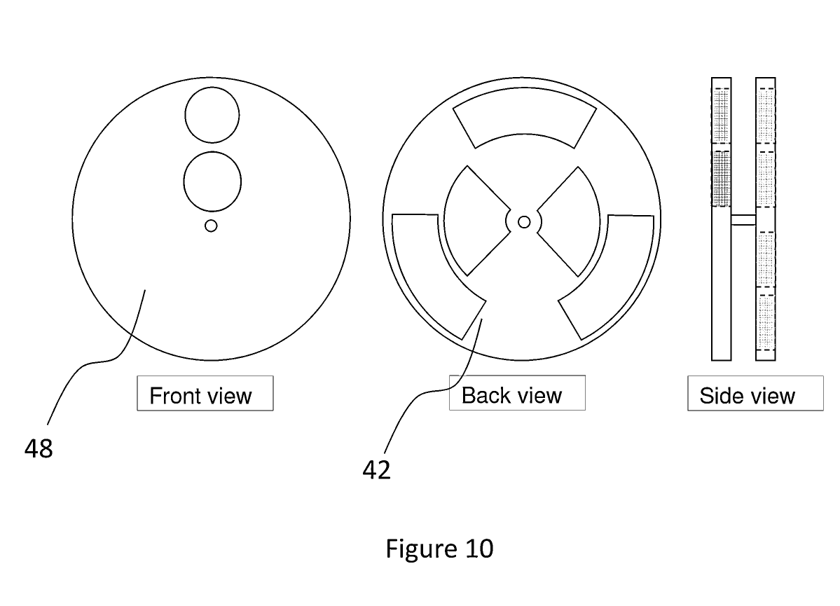

Referring to FIG. 10, another configuration of the wheel modulation is shown. In this configuration, two components, a static disc 48 and a wheel 42, are used and they are contained in the light modulation unit 20. The static disc 48 has color filters thereon which only allow light with certain wavelengths to pass through while the wheel 42 contains at least one hollow area for allowing light to pass through. The static disc 48 is coupled to the wheel 42. The light modulation unit 20 rotates the wheel 42 at a predetermined frequency and keeps the static disc 48 stationary so that the color filters on the static disc 48 and the hollow areas on the wheel 42 align along an axis intermittently. By doing so, the light signals are intermittently transmitted and blocked and thus are modulated at predefined frequencies. Similarly, the type, number and position arrangement of the color filters can be selected and the shape, number and position arrangement of the hollow areas can also be selected based on different requirements.

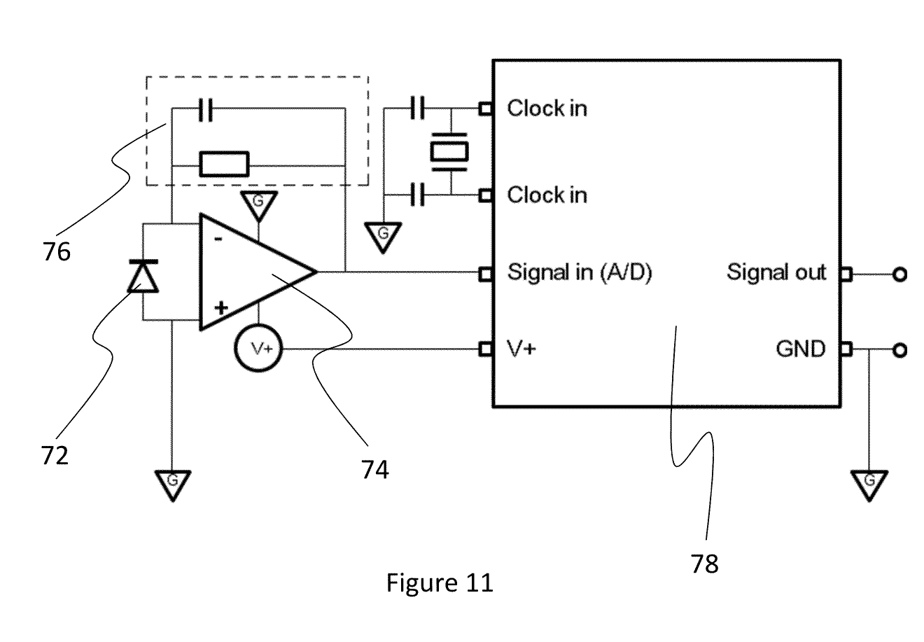

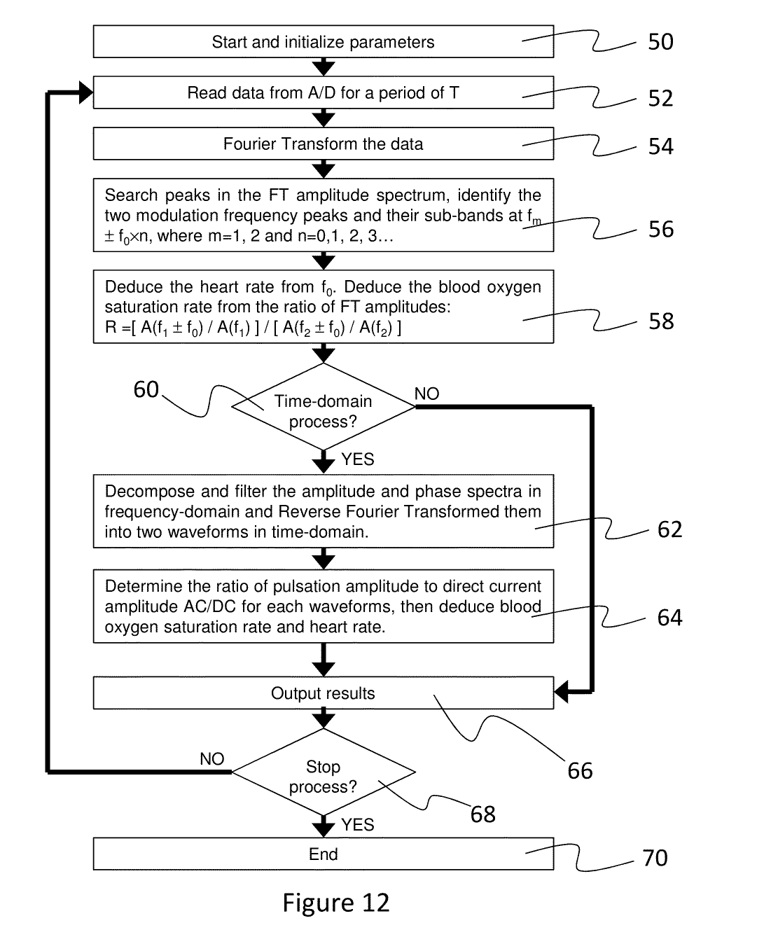

Now turning to FIG. 11, a simple circuit that implements the optical detection unit and the signal processing unit of the present invention is shown according to an embodiment. The photo-diode 72 converts the composite spectrophotometric light signal into an electrical signal. The operational amplifier 74, together with the RC high-pass filter circuit 76, further amplifies the electrical signal. Since the RC high-pass filter is in the feedback path of the operational amplifier, the output, amplified signal is low-pass filtered which will filter out wideband noise. This is fed to the ‘Signal In’ pin of the integrated circuit (IC) 78. Inside the integrated circuit 78, there is an analog to digital (A/D) converter that converts the amplified signal to digital signal, a central processor unit (CPU) and memory. The memory stores an embedded program that implements the flow chart to be described in FIG. 12. As the present invention implements the FT lock-in algorithm and analyzes the physiological data in the digital domain, only minimum analog circuitry is needed. Thus large scale integration IC can be used and the component count to realize the entire electrical circuitry can be minimized.

In FIG. 12, a flow chart of the Fourier Transform based lock-in detection algorithm of the present invention is shown. This flow chart is designed for measuring the heart rate and the blood oxygen saturation level. However, it can be easily adapted to measure other physiological data. The initialization step 52 is to start and initialize parameters such as modulation frequency, photo detector sensitivity, gain amplification, sampling frequency, sampling duration, A/D resolution, computation speed, memory capacity, etc. The signal processing unit 28 then reads data from the analog to digital convertor for a period of T in step 52. The sample frequency should be at least higher than the Nyquist rate of the highest modulation frequency; and is preferably 5 times the highest modulation frequency. In one embodiment, the sampling frequency is 2 KHz and T is around 10 seconds. In step 54, Fast Fourier Transform is performed to obtain a frequency spectrum of the data. In step 56, the signal processing unit 28 performs searching on the frequency spectrum so as to identify the dominant spectral peak and the minor peaks in the sub-bands at frequency (fₘ±f₀×n), where m=1, 2 and n=0, 1, 2, 3, etc. The process then goes to step 58 which calculates the physiological data such as heart rate f₀ and oxygen saturation rate according to eq. (5), etc. In step 60, the process will go to different states, depending on the selection of the user and the actual requirements. If time domain processing is needed, it goes to step 62, otherwise it goes to step 66. In the situation that a real time waveform is needed or additional information is needed, the time domain processing is required. For example, arterial stiffness index can be measured directly from the original PPG waveform. In step 62, the magnitude and phase spectra in the frequency domain are decomposed and filtered into two data sequences, each corresponding to a dominant spectral peak. Then the Inverse Fast Fourier Transform (IFFT) is performed for each of them to obtain two time domain waveforms. Based on the time domain waveforms, step 64 is to calculate the ratio of pulsation amplitude to direct current amplitude (AC/DC) for each waveform. Then the blood oxygen saturation rate and heart rate can then be deduced. In step 66, the calculation results are outputted on the display unit 30 or transmitted to any external devices 32. Then the program checks the ‘Stop process’ flag in step 68. If it is set, the whole process ends at step 70. Otherwise, program control loops back to step 52 to repeat the process again.

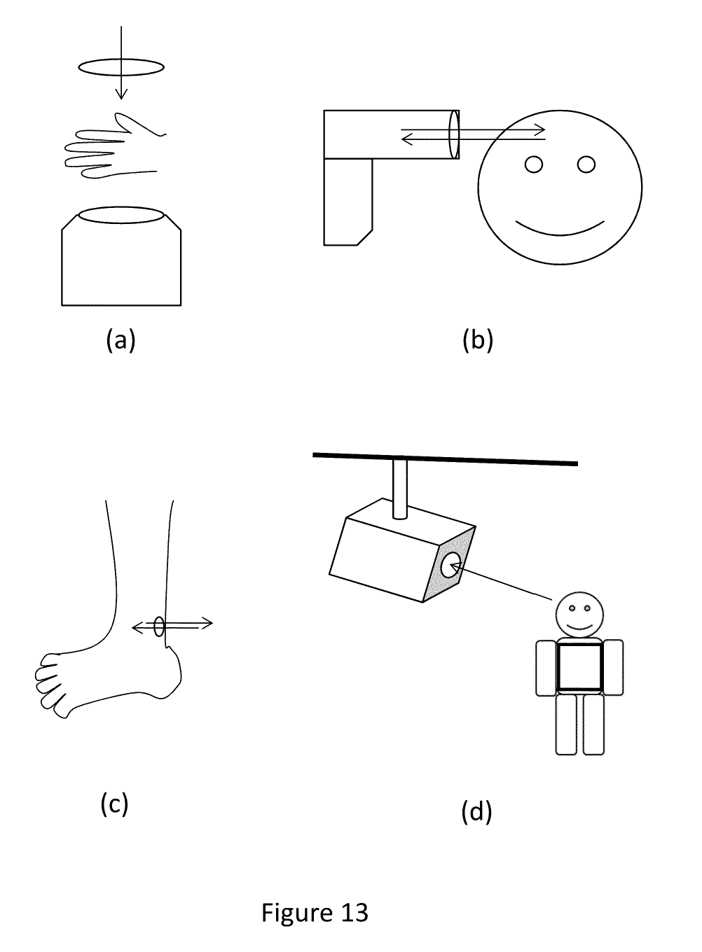

As mentioned before, not only can the present invention function in the conventional contact-mode of operation, but also be adapted for non-contact type of configurations. FIG. 13 illustrates several embodiments that the present invention can be configured to operate at different environments. FIG. 13a shows the microscope-type configuration of the present invention. Light bulbs or LEDs can be used as the light sources while a charge-couple device (CCD) camera or a photo-diode circuit can be used in the optical detector. The appendage of the subject 22 (typically the finger) can be placed in between for measurement. The apparatus can be designed in such a way that the finger does not contact either the light source(s) or the optical detector. Hence no contamination will arise. A gun type configuration of the present invention is shown on FIG. 13b. This gun type apparatus can be operated by a medical personal at a certain distance from a subject. On top of measuring heart rate and blood oxygen saturation, it can also combine with the use of IR thermometer to achieve multiple functions. The sensing area of this type is usually large. FIG. 13c shows a fiber-type configuration of the present invention in which it is flexible to be used on any parts of the body of the subject 22 due to the small size. The sensing head can be implemented by fiber tip. Finally, a telescope-type configuration of the present invention is shown in FIG. 13d. In this case, the present invention is capable of remote sensing which is non-disturbing to the subject 22. In principle, all four types of configuration can be designed to operate in either active mode or passive mode. However, actual engineering design and precision requirements may dictate whether active light sources are required. The telescope-type configuration as shown in FIG. 13d, however, is more amiable to passive mode of non-contact operation. The distance between the optical detection unit and the subject's skin varies according to different operating configurations. It ranges from 1 mm for the microscope-type configuration to 20 m for the telescope-type configuration. This range, however, is just exemplary figures and should not be construed as limits that this invention can achieve.

The exemplary embodiments of the present invention are thus fully described. Although the description referred to particular embodiments, it will be clear to one skilled in the art that the present invention may be practiced with variation of these specific details. Hence this invention should not be construed as limited to the embodiments set forth herein.

For example, the shape of the color filters can be of the shapes circle, triangle, square, rectangle, etc.

While heart rate and blood oxygen saturation parameters are used extensively in previous paragraphs to illustrate how the present invention can measure them effectively, the principle inventive ideas disclosed here can also be applied to measure other physiological data, including, but not limited to, heart rate variability, respiratory information, haemoglobin level, arterial stiffness index, cardiac output, etc. It should be noted that different number of light sources emitting light with different wavelengths may be needed to measure the aforementioned physiological signals.

As for the optical detection unit, although photo-diode and CCD array are mentioned in previous paragraphs, other apparatus such as photomultiplier tube, CMOS array or similar devices that can cover light signal into electrical signal can also be used.

What is claimed is:

1. A device for measuring physiological data of a subject comprising:
a) a light modulation unit for generating at least one modulated light signal by modulating at least one light source at at least one predefined frequency, said at least one modulated light signal being generated before irradiation onto said subject and each said at least one modulated light signal having a different wavelength;
b) an optical detection unit for receiving a composite spectrophotometric light signal and converting said composite spectrophotometric light signal into an electrical signal, said composite spectrophotometric light signal being generated when a physiological signal of said subject is regulated by said at least one modulated light signal; and
c) a signal processing unit adapted for:
i) converting said electrical signal into a digital signal;
ii) obtaining a frequency spectrum of said digital signal;
iii) tracking at least one dominant spectral peak of said frequency spectrum, each said dominant spectral peak corresponding to each said at least one predefined frequency;
iv) recognizing at least one minor spectral peak at said frequency spectrum from the vicinity of said at least one dominant spectral peak; and
v) determining said physiological data from said at least one dominant spectral peak and said at least one minor spectral peak.

2. The device of claim 1 wherein said light modulation unit comprises an electronic circuit that turns said at least one light source on and off at said at least one predefined frequency.

3. The device of claim 1, wherein said at least one modulated light signal further comprises a first modulated light signal emitting red light at a first predefined frequency and a second modulated light signal emitting infra-red light at a second predefined frequency.

4. The device of claim 1 wherein said light modulation unit comprises a wheel, said wheel being opaque to light and comprising at least one hollow area for allowing light to pass through, said wheel rotating at a predetermined frequency so that light emitted from said at least one light source is intermittently transmitted and blocked and thus is modulated at said at least one predefined frequency, said predefined frequency being an integer multiple of said predetermined frequency.

5. The device of claim 4 wherein said at least one hollow area being coupled to a color filter that only allows light with certain wavelength to pass through.

6. The device of claim 5 wherein said at least one light source being ambient light.

7. The device of claim 1 wherein said light modulation unit comprising:
a) a static disc with at least one color filter thereon which only allows light with certain wavelength to pass through; and
b) a wheel with at least one hollow area;
said static disc being coupled to said wheel, said wheel rotating at a predetermined frequency and said static disc being stationary in which said at least one color filter and said at least one hollow area align along an axis intermittently when said wheel rotates so that light emitted from said at least one light source is intermittently transmitted and blocked and thus is modulated at said at least one predefined frequency, said predefined frequency being an integer multiple of said predetermined frequency.

8. The device in claim 1 wherein said signal processing unit further comprises an analog-to-digital convertor, a central processing unit and memory, said processing unit executing an embedded program stored in said memory for determining said physiological data.

9. The device of claim 1, wherein said physiological data being heart rate, heart rate variability, respiratory information, haemoglogin level, arterial stiffness index, cardiac output, oxygen saturation of the blood, or any combination thereof.

10. The device of claim 1 further comprising an optical collection unit for said device to operate in a non-contact environment; said optical collection unit being an optical fiber, a lens, a mirror or a waveguide for directing said composite spectrophotometric light signal to said optical detection unit.

11. The device of claim 1 wherein said optical detection unit comprises a device selected from a photodiode, a photomulitplier tube, a CMOS array and a CCD array.

12. An apparatus for measuring physiological data of a subject comprising:
a) a light modulation unit for generating at least one modulated light signal by modulating an ambient light reflected from said subject at at least one predefined frequency, each said at least one modulated light signal having a different wavelength;
b) an optical detection unit for receiving a composite spectrophotometric light signal and converting said composite spectrophotometric light signal into an electrical signal, said composite spectrophotometric light signal being generated when a physiological signal of said subject is regulated by said at least one modulated light signal;
c) a processing unit for determining said physiological data from said electrical signal.

13. The apparatus of claim 12, wherein said light modulation unit comprises a wheel which is opaque to light, said wheel comprising at least one hollow area coupled to a color filter for allowing light with certain wavelength to pass through, said wheel rotating at a predetermined frequency so that said ambient light being modulated at said at least one predefined frequency, said predefined frequency being an integer multiple of said predetermined frequency.

14. The apparatus of claim 12, wherein said light modulation unit comprising:
a) a static disc with at least one color filter thereon which only allows light with certain wavelength to pass through; and
b) a wheel with at least one hollow area;
said static disc being coupled to said wheel, said wheel rotating at a predetermined frequency and said static disc being stationary in which said at least one color filter and said at least one hollow area align along an axis intermittently when said wheel rotates so that said ambient light being modulated at said at least one predefined frequency, said predefined frequency being an integer multiple of said predetermined frequency.

15. The apparatus of claim 12, wherein said processing unit is adapted for:
a) converting said electrical signal into a digital signal;
b) obtaining a frequency spectrum of said digital signal;
c) tracking at least one dominant spectral peak at said frequency spectrum, each said dominant spectral peak corresponding to each said at least one predefined frequency;
d) recognizing at least one minor spectral peak at said frequency spectrum from the vicinity of said at least one spectral peak; and
e) determining said physiological data from said at least one dominant spectral peak and said at least one minor spectral peak.

16. A method for determining physiological data of a subject comprising:
a) generating at least one modulated light signal by modulating at least one light source at at least one predefined frequency, each said at least one modulated light signal having a different wavelength;
b) detecting a composite spectrophotometric light signal and converting said composite spectrophotometric light signal into an electrical signal, said composite spectrophotometric light signal being generated when a physiological signal of said subject is regulated by said at least one modulated light signal;
c) converting said electrical signal into a digital signal;
d) obtaining a frequency spectrum of said digital signal;
e) tracking at least one dominant spectral peak at said frequency spectrum, each said dominant spectral peak corresponding to each said at least one predefined frequency;
f) recognizing at least one minor spectral peak at said frequency spectrum from the vicinity of said at least one dominant spectral peak; and
g) determining said physiological data from said at least one dominant spectral peak and said at least one minor spectral peak.

17. The method of claim 16, wherein said step (a) comprises turning a first light source on and off at a first frequency to emit red light and turning a second light source on and off at a second frequency to emit infra-red light.

18. The method of claim 16, wherein said step (a) comprises rotating a wheel at a predetermined frequency so that said at least one light source being modulated at said at least one predefined frequency, said wheel being opaque to light and comprising at least one hollow coupled to a color filter for allowing light with certain wavelength to pass through, said predefined frequency being an integer multiple of said predetermined frequency.

19. The method of claim 16, wherein said step (a) comprises rotating a wheel at a predetermined frequency and keeping a static disc coupled to said wheel stationary for modulating said at least one light source; said static disc comprising at least one color filter thereon which only allows light with certain wavelength to pass through; said wheel comprising at least one hollow area in which said at least one color filter and said at least one hollow area align along an axis intermittently when said wheel rotates, said predefined frequency being an integer multiple of said predetermined frequency.

20. The method of claim 16 wherein said determining step further comprises the step of computing the average of a first minor spectral peak and a second minor spectral peak; said first minor spectral peak being at the upper side band of said at least one dominant spectral peak and said second minor spectral peak being at the lower side band of said at least one dominant spectral peak.
